(19) United States
(12) Patent Application Publication
(10) Pub. No.: US 2010/0285576 A1
(43) Pub. Date: Nov. 11, 2010

(54) Method to produce synthesis gas or liquid fuels from commingled algae and coal feedstock using a steam-hydrogasification reactor and a steam methane reformer with CO2 utilization through an algae farm

(76) Inventors: Joseph M. Norbeck, Palm Desert, CA (US); Chan Seung Park, Yorba Linda, CA (US); Arun SK Raju, Riverside, CA (US)

Correspondence Address: BERLINER & ASSOCIATES, 555 WEST FIFTH STREET, 31ST FLOOR, LOS ANGELES, CA, 90013, US

(21) Appl. No.: 12/799,381

(22) Filed: Apr. 23, 2010

Related U.S. Application Data
(63) Continuation-in-part of application No. 10/911,348, filed on Aug. 3, 2004, now Pat. No. 7,500,997.
Application No. 10/911,348 is a continuation-in-part of application No. 10/911,348, filed on Aug. 3, 2004, now Pat. No. 7,500,997.
Application No. 10/911,348 is a continuation-in-part of application No. 10/503,435, filed on Jun. 28, 2005, now Pat. No. 7,208,530.
Application No. 10/503,435 is a continuation-in-part of application No. 11/879,241, filed on Jul. 16, 2007.
Application No. 11/879,241 is a continuation-in-part of application No. 11/489,298, filed on Jul. 18, 2006, now abandoned.
Application No. 11/489,298 is a continuation-in-part of application No. 11/879,266, filed on Jul. 16, 2007.
Application No. 11/879,266 is a continuation-in-part of application No. 11/489,308, filed on Jul. 18, 2006, now abandoned.
Application No. 11/489,308 is a continuation-in-part of application No. 12/286,165, filed on Sep. 29, 2008.
Application No. 12/286,165 is a continuation-in-part of application No. 11/879,456, filed on Jul. 16, 2007.
Application No. 11/879,456 is a continuation-in-part of application No. 11/489,299, filed on Jul. 18, 2006, now abandoned.
Application No. 11/489,299 is a continuation-in-part of application No. 12/218,653, filed on Jul. 16, 2008.
Application No. 12/218,653 is a continuation-in-part of application No. 11/879,267, filed on Jul. 16, 2007, now Pat. No. 7,619,012.
Application No. 11/879,267 is a continuation-in-part of application No. 11/489,353, filed on Jul. 18, 2006, now abandoned.
Application No. 11/489,353 is a continuation-in-part of application No. 11/635,333, filed on Dec. 6, 2006.
(60) Provisional application No. 61/172,176, filed on Apr. 23, 2009.

(51) Int. Cl. C07C 1/02; C07C 27/06; C12M 1/04; C10G 1/06

(52) U.S. Cl. 435/303.1; 252/373; 518/704; 208/414

(57) ABSTRACT

This invention involves the conversion of coal-algae or resid-algae commingled slurry feedstock into a high methane content product gas using a steam hydrogasification process. This gas is then reformed into synthesis gas (H₂ and CO). Excess H₂ from the synthesis gas is separated and recycled back to the gasifier. The synthesis gas is converted into a liquid fuel such as Fischer-Tropsch diesel. The CO₂ emissions from the steam hydrogasification process can be captured and used to grow the algae, which can subsequently be commingled with coal or reside to form slurry feedstocks for the hydrogasifier. Thus, this process eliminates CO₂ emissions from the conversion plant.

### CROSS-REFERENCE TO RELATED APPLICATIONS

This application claims the benefit of the provisional application 61/172,176 filed on Apr. 23, 2009, and

is a continuation-in-part of, and claims the benefit of, patent application Ser. No. 10/911,348, filed Aug. 3, 2004, which is a continuation-in-part of, and claims the benefit of U.S. Pat. No. 7,208,530 which was reissued as RE40419, which claims the benefit of Provisional application 60/355,405, filed Feb. 5, 2002;

is a continuation-in-part of, and claims the benefit of, patent application Ser. No. 11/879,241, filed Jul. 16, 2007, which is a continuation-in-part of, and claims the benefit of, patent application Ser. No. 11/489,298, filed Jul. 18, 2006;

is a continuation-in-part of, and claims the benefit of, patent application Ser. No. 11/879,266, filed Jul. 16, 2007, which is a continuation-in-part of, and claims the benefit of, application Ser. No. 11/489,308, filed Jul. 18, 2006;

is a continuation-in-part of, and claims the benefit of, patent application Ser. No. 12/286,165, filed Sep. 29, 2008, which is a continuation-in-part of, and claims the benefit of, application Ser. No. 11/879,456 filed Jul. 16, 2007, which is a continuation-in-part of, and claims the benefit of, application Ser. No. 11/489,299 filed July 18;

is a continuation-in-part of, and claims the benefit of, patent application Ser. No. 12/218,653, filed Jul. 16, 2008, which is a continuation-in-part of, and claims the benefit of patent application Ser. No. 11/879,267, filed Jul. 16, 2007, which is a continuation-in-part of, and claims the benefit of, application Ser. No. 11/489,353, filed Jul. 18, 2006; and

is a continuation-in-part of, and claims the benefit of, patent application Ser. No. 11/635,333, filed Dec. 6, 2006.

### STATEMENT REGARDING SPONSORED RESEARCH OR DEVELOPMENT

This invention was made with support from the City of Riverside, Calif. The City of Riverside has certain rights in this invention.

The disclosures of the above cited applications are all incorporated herein by reference in their entirety.

### FIELD OF THE INVENTION

The invention relates to a steam hydrogasification process and apparatus utilizing commingled algae-carbonaceous material to generate synthesis gas or liquid fuels.

### BACKGROUND OF THE INVENTION

Steam hydrogasification (SHR) based gasification processes have been previously described in detail in Norbeck et al. U.S. patent application Ser. Nos. 10/503,435 (published as US 2005/0256212), and 10/911,348 (published as US 2005/0032920). The disclosure of U.S. patent application Ser. Nos. 10/503,435 and 10/911,348 are incorporated herein by reference in their entirety. Such processes can occur in the absence of catalysts, injection of air, oxygen (i.e. partial oxidation conditions), hot solids, or other initiating chemicals. In this steam hydrogasification process, the carbonaceous feedstock is first converted to a fuel gas, containing a significant quantity of methane. The fuel gas in the next step is then reformed to generate synthesis gas (carbon monoxide and hydrogen) in a Steam Methane Reformer (SMR). In the third step, the synthesis gas is converted into a synthetic fuel over a high-efficiency catalyst. Examples of such synthetic fuels are Fischer-Tropsch (FT) diesel, methanol, dimethyl ether (DME), etc. The production of high energy density liquid fuels such as the FT diesel is desirable from a fuel handling and distribution perspective. A process flow diagram of this technology is shown below.

As shown in FIG. 1, SHR is the hydrogasification reaction carried out in the presence of steam. The SHR step is followed by the Steam Methane Reforming (SMR) step to produce the syngas. The hydrogen necessary for the SHR is generated internally and is recycled back to the SHR.

The SHR step utilizes a water based slurry as the source of carbonaceous feedstock and combines it with steam and recycled hydrogen to produce a methane rich gas. The reactions of the carbonaceous slurry feedstock in the SHR can be chemically represented in a simplified manner as:

C+H₂O+2H₂→CH₄+H₂O+Others (1)

The SMR that converts products formed in reaction (1) into synthesis gas can be characterized as:

CH₄+others+H₂O→3H₂+CO+CO₂ (2)

It is important to note that the SMR step requires high temperature steam together with methane rich gas to produce the synthesis gases. Thus, there is no need to remove the steam from the SHR product gas stream after the reactor. The introduction of water in the form of slurry into the SHR reactor is one of the most unique features of our SHR process. Water acts as the carrying medium for the carbonaceous feedstock into the SHR by utilizing a conventional slurry pumping technology. It also enhances the product gas yield as well as the reactivity of the hydrogasification process. Water is consumed by the SMR (in the form of steam) as a feedstock to produce the synthesis gas. SHR feedstocks with high moisture content such as biomass or biosolids can be directly mixed with other feedstocks such as coal. This avoids the feedstock drying expenses faced by other dry feed technologies.

The SMR produces a syngas with a H₂/CO ratio higher than the value required by the Fischer-Tropsch process. The excess hydrogen of the SMR product gas can then be separated and fed back to the SHR, making the process self sustained (i.e., no need for an external source of hydrogen after initial start up).

Synthetic fuel (methanol, DME or FT diesel) is generated from the synthesis gas made in the SHR & SMR reactors coupled with a warm gas cleanup unit. Details of the gas clean up unit have been described previously in patent application Ser. No. 11/879,266, filed Jul. 16, 2007; application Ser. No. 11/489,308, filed Jul. 18, 2006; and patent application Ser. No. 11/635,333, filed Dec. 6, 2006, the details of which are all herein incorporated by reference in their entirety.

### BRIEF SUMMARY OF THE INVENTION

A method of using algae in an algae farm as slurry feedstock for steam hydrogasfication and to capture carbon dioxide emissions during liquid fuel production is provided that involves providing a slurry feedstock to a hydrogasification reactor; heating the slurry feedstock with hydrogen, at a temperature and pressure sufficient to generate a stream of methane and carbon monoxide rich gas product; subjecting the resultant producer gas to steam methane reforming under conditions whereby synthesis gas comprising hydrogen, carbon monoxide and carbon dioxide is generated; providing an algae farm, and feeding the algae farm with carbon dioxide generated from said steam reforming.

In another embodiment, a steam hydrogasification process is provided that combines the use of an algae farm and a direct coal liquefaction process, where resid generated by the liquefaction process can be commingled with algae to feed the steam hydrogasifier.

In yet another embodiment, a steam hydrogasification process is provided that combines the use of an algae farm and a direct coal liquefaction process, where resid generated by the liquefaction process can be commingled with algae to feed the steam hydrogasifier, and hydrogen generated by a steam methane reformer is fed into the liquefaction process.

The present invention is advantageous because it provides a flexible steam hydrogasification process that can 1) utilize algae farms to form coal or resid-algae slurries as feedstock for steam hydrogasification; 2) utilize algae farms to capture carbon dioxide generated by the steam hydrogasification process; and 3) generate hydrogen that can be fed to a direct coal liquefaction process.

### BRIEF DESCRIPTION OF THE DRAWINGS

For a more complete understanding of the present invention, reference is now made to the following descriptions taken in conjunction with the accompanying drawing, in which:

FIG. 1 shows a flow diagram of the steam hydrogasfication process.

FIG. 2 shows a flow diagram of the steam hydrogasfication process utilizing an algae farm.

FIG. 3 shows a flow diagram of the steam hydrogasification process with an algae farm in conjunction with a Direct Coal Liquefaction process.

### DETAILED DESCRIPTION OF THE INVENTION

A process of using algae farms as part of a steam hydrogasification process is provided. The advantages of the process are that the CO₂ generated by hydrogasfication process can serve as a CO₂ supply feed for the algae farms; and algae subsequently grown by this CO₂ can then serve as the principal, or part of the, feedstock for the same hydrogasification process.

The steam hydrogasification process can occur in the absence of catalysts, injection of air, oxygen (i.e. partial oxidation conditions), hot solids, or other initiating chemicals.

Algae farms, as the term used a here, is defined as any space or location where algae can be cultivated. These locations can include, for example, enclosed spaces or reactors, or combinations thereof.

The CO₂ emissions from the steam hydro-gasification process, as disclosed, is captured and is used as a feed to grow high growth rate biomass such as algae in a high efficiency algae bioreactor. A number of these bioreactors together can serve as an algae farm. Thus the CO₂ can be converted into algae which in turn can be converted into energetic products as a result of feeding the algae into the steam hydrogasification (or direct coal liquefaction) process. Although algae farms as a means to utilize CO₂ have been proposed before, currently studied pathways to utilize the algae crop involve producing biodiesel from algae triglyceride oil. However the overall efficiency of such processes is much lower than that of thermo-chemical processes (˜15 to 18% less than thermo-chemical processes in general) due to multiple steps involved and limited feedstock utilization. Thus, in one embodiment a process is provided that utilizes algae to produce synthetic fuels/biodiesel from a hydrogasification process, without processing of/utilizing the algae triglyceride oil.

Algae farms can potentially be used as a source of a significant feedstock for this SHR process since the SHR gasifier can accept algae as a feed along with other conventional feedstocks such as coal. Indeed, one major advantage of the present SHR process is that the process can accept feedstock with a high water content (i.e. in the form of slurries). For instance, water to carbon ratios in the range of about 0.5:1 to 4:1 (preferably 1:1 to 3:1) can be used in the SHR. The SHR process is able to utilize the water content within the algae plant itself (or the water serving as the environment for the algae crop) to form a coal-algae slurry feed for the SHR. In one embodiment, the water content of the algae plant itself (or the environment around the crop) can serve as the sole/only source of water feed for the SHR. In another embodiment, the SHR can be fed with water supplied/generated from a combination of the algae plant itself/algae crop environment and another source. Water to create the coal-algae slurries to feed the SHR can also be obtained from a Fischer Tropsch reactor, which can be utilized downstream after the SMR in the same process.

Here, water acts as the carrying medium for the carbonaceous feedstock into the SHR by utilizing a conventional slurry pumping technology. It also enhances the product gas yield as well as the reactivity of the hydrogasification process. The water, as part of the slurry, is also later consumed by the SMR (in the form of steam) as a feedstock to produce the synthesis gas. In one embodiment, the steam and the methane produced by the SHR can serve as the sole/only source of feed for the SMR for the production of synthesis gas. In another embodiment, the SMR can be fed with steam and/or methane supplied/generated from a combination of the SHR and a non-SHR source (i.e. steam produced from a steam generator; or methane generating process known in the art).

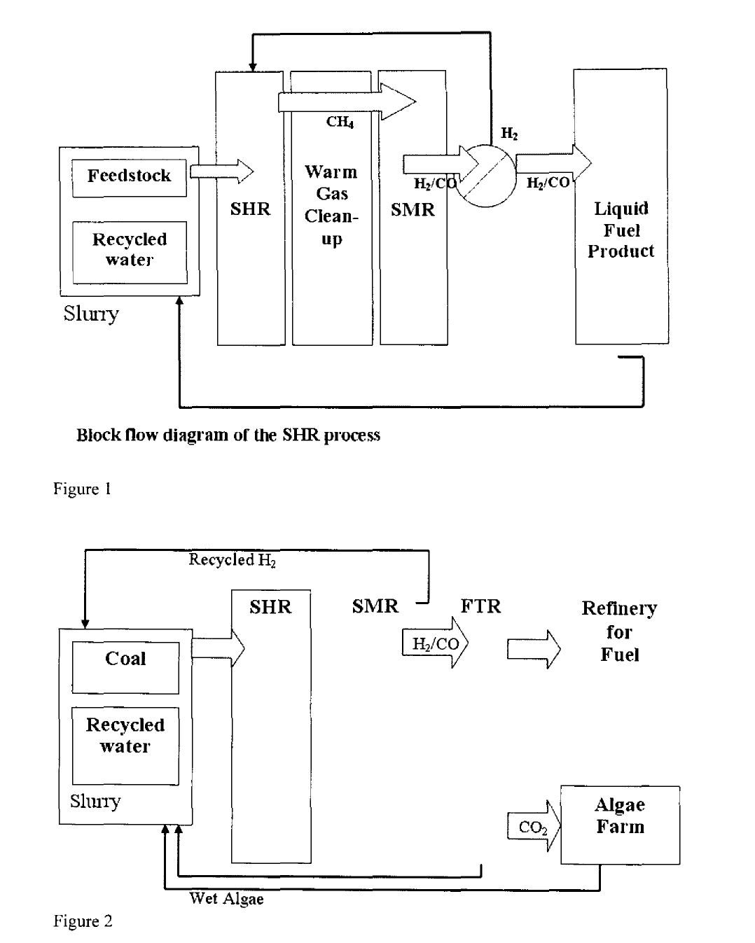

The steam hydrogasification process utilizing the algae farm is shown in FIG. 2. Coal is co-mingled with the wet algae from the algae farm to form a coal-algae slurry feedstock for the SHR. Process CO₂ released from either one or both the SMR and FTR can be captured by Flexsorb process (not shown) and this CO₂ can serve as the only/sole CO₂ feed for the algae farm. Thus, CO₂ emissions from the steam hydrogasification process are negligible. In another embodiment, the algae farm can be feed CO₂ from a combination of the SMR and FTR, as well as other sources/processes.

The hydrogen generated by the SMR can be recycled and serve as the sole/only source of hydrogen feed for the SHR. In another embodiment, the hydrogen generated by the SMR can be recycled and serve as the sole/only source of hydrogen feed for the SHR once the hydrogasfication process has been initiated utilizing a external source of hydrogen.

In another embodiment, the SMR can be fed with hydrogen supplied/generated from a combination of both SMR and a non-SMR source (i.e. a hydrogen generating device/process known in the art).

### Alternative Embodiment with Direct Coal Liquefaction

It is well known that Direct Coal Liquefaction (DCL) processes require hydrogen and generate a high carbon content waste known as ‘resid’ in addition to the coal based liquid. Apparatus used for such DCL associated processes are also well known in the art. In another embodiment of the invention, the above hydrogasification process utilizing algae farms can also be used in conjunction with a DCL process. In this embodiment the DCL generated ‘resid’ can be combined with wet algae (from the algae farm) to form the slurry feedstock for the SHR.

In one embodiment, the slurry feedstock comprising of resid and algae can be processed using steam hydrogasification, steam methane reformation and Fischer-Tropsch reactors to produce liquid fuels or heat.

In one embodiment, the water content of the algae plant itself (or the environment around the crop) can serve as the sole/only source of water to form the resid slurry. In another embodiment, the water to create the resid/coal-algae slurries to feed the SHR can also be obtained from a Fischer Tropsch reactor, an optional part of the process, or other sources.

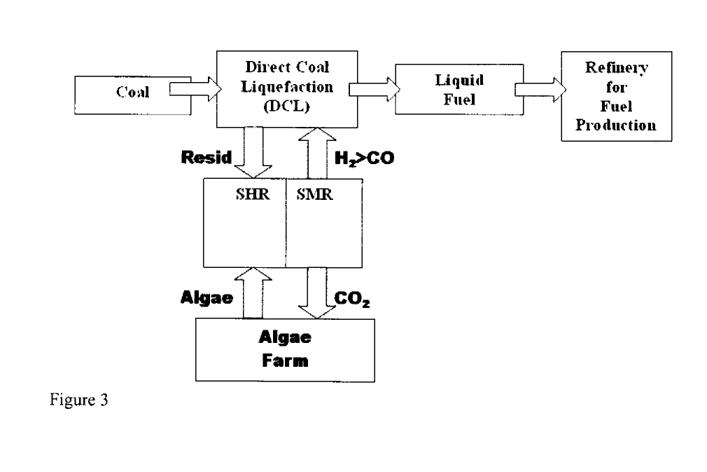

The slurry feedstock comprising of resid and algae can be processed using steam hydrogasification and steam methane reformation (see FIG. 3). Here, hydrogen generated from the SMR can serve as sole/only source of hydrogen feedstock for the DCL process. In another embodiment, the DCL process can also utilize hydrogen from additional sources. The carbon dioxide generated from the SMR syngas can serve as the sole/only CO₂ feed, or as part of the total CO₂ feed, for the algae farm, which in turn results in the production of algae that can serve as the feedstock for the SHR reactor. This particular embodiment solves multiple problems concerning providing a H₂ supply for a DCL processes; capturing the CO₂ released from the SMR and DCL; and providing a water source to be combined with resid to form feedstock slurries for the SHR. Thus, in one embodiment, a hydrogasification process is disclosed that utilizes solely/only on the recycled H₂, CO₂, and water. In another embodiment, a hydrogasification process is disclosed that utilizes solely/only on the recycled H₂, (once the hydrogasfication process has been initiated utilizing a external source of hydrogen), CO₂, and water from said process.

In another embodiment of the invention, a hydrogasification apparatus comprising a hydrogasifier, a steam methane reformer, and an algae farm is provided. In a more particular embodiments, gas clean up units and/or a Fischer-Tropsch reactor are provided. In yet another embodiment of the invention, a hydrogasification apparatus comprising a hydrogasifier, a steam methane reformer, an algae farm and DCL associated apparatus are provided. In yet another embodiment, the provided apparatus comprising a hydrogasifier, a steam methane reformer, an algae farm and DCL associated apparatus are able to run solely/only on recycled H₂ (or optionally some initial external source of H₂ to initiate the process), CO₂, and water produced from said apparatus itself.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the following claims.

1. A process of using algae from an algae farm as slurry feedstock for hydrogasfication and to capture carbon dioxide emissions during liquid fuel production comprising:
providing a slurry feedstock to a hydrogasification reactor;
heating the slurry feedstock with hydrogen, at a temperature and pressure sufficient to generate a stream of methane and carbon monoxide rich gas product;
subjecting the resultant producer gas to steam methane reforming under conditions whereby synthesis gas comprising hydrogen, carbon monoxide and carbon dioxide is generated;
providing an algae farm, and
feeding the algae farm with the carbon dioxide generated from said steam reforming.

2. The process of claim 1, further comprising feeding the hydrogasfication reactor with the algae from the algae farm.

3. The process of claim 1, wherein additional steam is used with hydrogen to heat the feedstock.

4. The process of claim 1, wherein the feedstock comprises of carbonaceous material and algae.

5. The process of claim 1, wherein the process is able to run solely/only on the H₂, CO₂, and water actually generated from the process itself.

6. The process of claim 1, wherein the carbonaceous material is selected from the group consisting of municipal waste, biomass, wood coal, or natural or synthetic polymer.

7. The process of claim 1, further comprising feeding the synthesis gas into a Fischer-Tropsch reactor whereby liquid fuel, carbon dioxide, and water is generated.

8. The process of claim 6, further comprising recycling the water generated by the Fischer-Tropsch reactor into the hydrogasification reactor.

9. The process of claim 1, further comprising a direct coal liquefaction process whereby liquid fuel and resid are generated.

10. The process of claim 9, further comprising feeding said resid into the hydrogasfication reactor.

11. The process of claim 9, wherein the hydrogen generated from the steam reforming is recycled back into the direct coal liquefaction process.

12. The process of claim 1, wherein the hydrogen generated from the steam reforming is recycled back into the steam hydrogasfication reactor.

13. The process of claim 9, wherein the hydrogen generated from the steam reforming is recycled back into the steam hydrogasfication reactor and direct coal liquefaction process.

14. The process of claim 1, wherein the hydrogasification process can occur in the absence of catalysts.

15. The process of claim 1, wherein the hydrogasification process can occur in the absence of oxygen.

16. A process for using algae in an algae farm comprising
growing the algae using CO₂ released from a syngas producing process,
and feeding the algae as part of a slurry feedstock into the syngas producing process.

17. An apparatus for hydrogasification of carbonaceous material comprising:
a hydrogasification reactor;
a gas clean up unit;
a steam methane reformer;
and an algae farm.

18. The apparatus of claim 16, further comprising apparatuses used in a DCL process.
